(19) United States
(12) Patent Application Publication
(10) Pub. No.: US 2009/0087912 A1
(43) Pub. Date: Apr. 2, 2009

(54) TAGGED PARTICLES FOR DOWNHOLE APPLICATION

(75) Inventors: Rogerio T. Ramos, Eastleigh (GB); Iain Cooper, Sugar Land, TX (US); J. Ernest Brown, Fort Collins, CO (US)

Correspondence Address: SCHLUMBERGER RESERVOIR COMPLETIONS, 14910 AIRLINE ROAD, ROSHARON, TX, 77583, US

(73) Assignee: SHLUMBERGER TECHNOLOGY CORPORATION, SUGAR LAND, TX (US)

(21) Appl. No.: 12/240,607

(22) Filed: Sep. 29, 2008

Related U.S. Application Data
(63) Continuation-in-part of application No. 11/863,598, filed on Sep. 28, 2007.
(60) Provisional application No. 60/990,642, filed on Nov. 28, 2007.

(51) Int. Cl. G01N 33/24

(52) U.S. Cl. 436/27; 436/29

(57) ABSTRACT

A tagged object includes a main body and a plurality of coded particles. Each coded particle may have a miniature body and be configured to provide a resolvable optical emission pattern when illuminate. The plurality of coded particles may be immobilized to the main body. A method for performing oilfield monitoring may include disposing of different types of tagged objects at different locations, wherein the different types of tagged objects each comprise a plurality of coded particles. Each of the coded particles may have a miniature body containing rare earth elements configured to produce a unique optical emission pattern when illuminated. The method may include allowing an event to trigger the release of one of the different types of tagged objects from one of the different locations. In addition, the method may include identifying the released tagged objects by unique optical emission patterns, in some cases in order to determining an occurrence location of the event.

### CROSS-REFERENCE TO RELATED APPLICATIONS

This application is a Continuation-In-Part of U.S. patent application Ser. No. 11/863,598, filed Sep. 28, 2007, and claims the benefit of U.S. Provisional Application Ser. No. 60/990,642, filed Nov. 28, 2007, both of which are incorporated herein by reference in their entirety.

### BACKGROUND OF INVENTION

1. Field of the Invention

The present invention relates generally to the field of tracers or marker materials. More specifically, the invention relates to subsurface tagging and monitoring techniques.

2. Background Art

Tracers have been used in the oil and gas industry for many years. One use of the tracers is to determine the “lag time” for the drilling fluid (“mud”) to travel from the surface down the borehole, through the drill bit, and up to the surface again. A conventional technique for this purpose involves the use of calcium carbide pellets that have been enclosed in a water-proof container. Such carbide pellets are injected from the surface, together with the mud stream, down the borehole. When these pellets pass through the drill bit, the water-proof container is smashed leading to the release of the calcium carbide, which then reacts with water in the mud to form a gas, acetylene. Acetylene, together with returning mud, rise to the surface and may be detected at the surface with a gas analyzer. The lag time may be determined from the time of injection of the calcium carbide into the well until the detection of gas at the surface in the return mud.

Another conventional use of tracers relates to the injection of tracers into a well, followed by their detection in the adjacent formation or in an adjacent well. Such techniques permit the assessment of well fluid invasion into the surrounding formations or permit well-to-well correlations to enable the characterization of underground formations between the two wells. Tracers for such uses may be radioactive substances or chemicals that can be readily identified.

For example, U.S. Pat. No. 4,447,340 describes a method of tracing well drilling mud penetration into formations by determining the concentration of acetate tracer ion in the penetrated strata (e.g., by core analysis). Other tracers useful for such analysis may include, for example, dichromate, chromate, nitrate, ammonium, cobalt, nickel, manganese, vanadium and lithium.

U.S. Pat. No. 5,243,190 describes the use of radioactive particles for subsurface tracers. The use of radioactive substances as tracers is not always been desirable due to safety and environmental considerations.

Other tracer techniques using spectroscopic techniques have also been proposed. Suitable spectroscopy may include atomic absorption spectroscopy, X-ray fluorescence spectroscopy, or neutron activation analysis, to identify certain materials as tagging agents. For example, U.S. Pat. No. 6,725,926 proposes the use of a proppant coated with phosphorescent, fluorescent, or photoluminescent pigments that glow in the dark upon exposure to certain lighting. Fluorescence spectrometry techniques entailing the illumination of fluids with a light source have also been proposed (See e.g., U.S. Pat. Nos. 7,084,392, 6,707,556, 6,564,866, 6,955,217, U.S. Patent Publication No. 20060054317).

These conventional tracer techniques have been quite useful. However, a need remains for improved tracer/tagging techniques, particularly in the areas of oil, gas, and water exploration and production.

### SUMMARY OF INVENTION

One aspect of the invention relates to tagged objects. A tagged object in accordance with one embodiment of the invention includes a main body and a plurality of coded particles. Each of the plurality of coded particle may have a miniature body and be configured to provide a resolvable optical emission pattern when illuminated. In addition, the plurality of coded particles may be immobilized to the main body.

Another aspect of the invention relates to systems for tagging. A system for tagging in accordance with one embodiment of the invention may include a plurality of tagged objects, wherein each of the plurality of tagged objects has a main body. In addition, the system may include a plurality of coded particles, wherein each coded particle may have a miniature body and be configured to provide a resolvable optical emission pattern when illuminated. The plurality of coded particles may be immobilized to the main body. The plurality of tagged objects may each have a different optical emission pattern.

Another aspect of the invention relates to methods for performing oilfield monitoring. A method in accordance with one embodiment of the invention may include disposing different types of tagged objects at different locations, wherein the different types of tagged objects each comprise a plurality of coded particles. Each of the plurality of coded particles may have a miniature body containing rare earth elements to produce a unique optical emission pattern when illuminated. The method may further include allowing an event to trigger a release of one of the different types of tagged objects from one of the different locations. In addition, the method may include identifying the released tagged objects by the unique optical emission patterns to determine an occurrence location of the event.

Other aspects and advantages of the invention will be apparent from the following description and the appended claims.

### BRIEF DESCRIPTION OF DRAWINGS

Other aspects and advantages of the invention will become apparent upon reading the following detailed description and upon reference to the drawings in which like elements have been given like numerals and wherein:

FIG. 1 is a schematic of particles revealing a coded pattern of fluorescence emission in response to illumination by a light source in accordance with aspects of an embodiment of the invention;

FIG. 2 is a schematic of a well drilling system including coded particle release units and a particle detection unit in accordance with aspects of an embodiment of the invention;

FIG. 3 is a schematic of a coded particle release unit in accordance with aspects of an embodiment of the invention;

FIG. 4 is a schematic of a downhole tool including coded particle release and detection units in accordance with aspects of an embodiment of the invention;

FIG. 5 is a schematic of another downhole tool including coded particle release units in accordance with aspects of an embodiment of the invention;

FIG. 6 is a schematic of a downhole tool including a coded particle release unit and implemented in a well-to-well application in accordance with aspects of an embodiment of the invention;

FIGS. 7A and 7B show schematics of tagged objects having a plurality of coded optical particles immobilized to the objects in accordance with aspects of embodiments of the invention;

FIG. 8 show a schematic of another tagged object having a plurality of coded optical particles immobilized to the object in accordance with one embodiment of the invention; and

FIG. 9 is a flow chart of a tagging method in accordance with aspects of an embodiment of the invention.

### DETAILED DESCRIPTION

Embodiments of the present invention relate to coded particle technology. Embodiments of the invention use small particles doped with different substances, such as rare earth elements, that can provide an unique optical emission when excited with a light source (e.g., with an appropriate wavelength radiation). In accordance with some embodiments of the invention, rare earth-doped glasses are chosen because of their narrow emission bands, high quantum efficiencies, non-interference with common fluorescent labels, and inertness to most organic and aqueous solvents. These properties and the large number of possible combinations of these microbarcodes make them attractive for use in subsurface or downhole applications.

Embodiments of the invention may be based in part on the coded particle technology described in M. J. Dejneka et al., Optically active glasses for biology, 3-D display, and telecommunications, Proceedings of the XX ICG International Congress on Glass, Kyoto, Sep. 27 to Oct. 1, 2004; M. J. Dejneka et al., Rare earth-doped glass microbarcodes, Proceedings of the National Academy of Sciences of the United States of America, PNAS, Jan. 21, 2003, vol. 100, no. 2, 389-393 (hereinafter “the Dejneka Papers”). These papers describe micrometer-sized glass barcodes containing a pattern of different fluorescent materials that are easily identified using a UV lamp and an optical microscope. These microbarcodes are suitable for complicated assays, such as those requiring multiple identification codes in a small volume. For example, the PNAS paper describes a model DNA hybridization assay using these “microbarcodes.”

Some embodiments of the invention relate to the use of micrometer-sized barcodes (microbarcodes) that contain patterns of different fluorescent materials as tracers in downhole applications. The patterns of different fluorescent materials are easily identified by illumination with light of certain wavelengths. For example, the patterns of fluorescent materials may ne identified using a UV lamp and an optical microscope.

In accordance with some embodiments of the invention, the micrometer-sized barcodes (microbarcodes) may use rare earth (RE) ions in a silicate glass matrix for example. Such materials are particularly suitable for the fabrication of encoded particles because the RE-doped glasses have narrow emission bands, high quantum efficiencies, noninterference with common fluorescent labels, and inertness to most organic and aqueous solvents. These properties and the large number of possible combinations (greater than 1 million, based on combination and permutations of various RE elements) of the microbarcodes make them attractive for use in subsurface encoding applications.

As described in the Dejneka Papers, REs are a spectroscopically rich species, which makes their use as optical codes in a spectral window distinct from conventional, organic fluorescent dyes. Due to their narrower spectral bands, REs allow more resolvable bands to be packed into the same spectral bandwidth; this enables a larger number of distinct combinations for coding applications. For example, multiple RE ions can be simultaneously excited in the UV spectrum and conveniently decoded by observing their emission in the visible region, without interfering with other materials that have excitations in the visible region. They are also resistant to photobleaching.

A silica-based glass matrix for the particles offers some advantages, including compatibility with organic solvents and low background fluorescence that provides lower limits of detection (i.e., enhanced sensitivity). Glass preforms may be drawn down to very thin fibers or ribbons, whose structures are an exact miniature of the parent preforms, allowing large complex structures to be replicated down to a desired size.

In accordance with embodiments of the invention, a method for fabricating glass particles comprises mixing RE-doped glass compositions (e.g., alkaline earth aluminosilicate glass composition), each with a unique RE (which may be in an oxide form, e.g., RE₂O₃) for a particular color. The compositions are then melted, cast into patties, and annealed. After these treatments, the patties, having different REs for different fluorescence colors, are then assembled in a selected order (bar codes) for producing a glass micobarcodes.

Conventional optical fiber draw methods may be used to fabricate encoded fiber ribbons. For example, the assembly of different glass patties having different REs is fused in a furnace and the preform is drawn into a ribbon fiber of suitable dimensions (e.g., 20 μm thick and 100 μm wide). The ribbon fiber is then scribed (e.g., every 20 μm) to “pre-score” the ribbon fiber, for example, by laser pulses controlled by a computer (i.e., a computer-controlled laser stage). The scribed ribbon fiber may then be sonicated in water to break the ribbon along the scribes into individual barcode pieces.

While the above description illustrates how microbarcodes of the invention may be fabricated, other suitable materials or methods may also be used without departing from the scope of the invention. That is, fabrication of RE-doped barcodes is not limited to the use of a silica-based matrix or fiber draw techniques, alternative approaches are possible using other materials and techniques. For example, the above described glass compositions may be prepared with a matrix based on any other type of glass, crystal glass, crystal, a type of silicon oxide, germanium oxide, aluminum oxide, boron oxide, ceramic, or polymer. In accordance with some embodiments of the invention, the particle matrix may comprise other components (such as a ferromagnetic material) to endow the particles with a desired property. For example, with an added ferromagnetic material, a magnetic field may be used to collect or extract the particles for analysis.

In accordance with some embodiments of the invention, the barcode particles may be decoded and imaged by using a spectral imager and a fluorescence microscope equipped with a proper light source (e.g., a mercury lamp). One skilled in the art would appreciate that any suitable light source with a proper filter may be used to produce the desired excitation wavelengths. For example, a dichroic filter may be used to provide an excitation wavelength from a mercury lamp. Similarly, a filter may be used to detect the fluorescence light (emission) generated by the barcode particles. For example, a 420-nm long-pass filter may be used to observe RE fluorescence. It may be appreciated by those skilled in the art that various combinations of filters and imaging equipment may be used with embodiments of the invention.

Suitable RE ions for preparing microbarcode particles of the invention preferably have non-overlapping, bright visible luminescence for ease of detection. For example, the Ce³⁺, Tm³⁺, Tb³⁺, and Dy³⁺-doped glasses glow cyan, blue, green, and pale orange/yellow, respectively, when excited with the proper wavelengths. These distinct light emissions may be detected by the naked eye or with a spectrometer or a microscope. In addition, in accordance with some embodiments of the invention, RE ions preferably have a common excitation wavelength range (say 250-350 nm) such that they may be simultaneous interrogated with a single light source. For example, a UV lamp may be used to achieve a multiplexed (simultaneous) excitation of different RE ions in the particles. A usable UV light source may be a mercury lamp (e.g., one that emits at 254 and 365 nm).

The coded particles of the invention may be configured with different numbers of barcodes by varying the number of bands in a ribbon. In addition, the intensities of the barcode signals may be varied by the dimensions of the particles (e.g., by different scribe-lengths of the ribbon) and/or the concentrations of the RE ions in the particles.

In accordance with embodiments of the invention, various coding schemes may be used. For example, some embodiments of the invention may involve a simple binary-type “yes/no” coding scheme, i.e., the presence or absence of a particular color (RE ion). Some embodiments of the invention may involve combinations of colors and/or sequences within a particle. For example, some embodiments may involve “binary combination of colors,” such as Ce³⁺—Tb³⁺, Ce³⁺—Dy³⁺, Tm³⁺—Tb³⁺, Tm³⁺—Dy³⁺, and Tb³⁺—Dy³⁺. These doped glasses produce clearly resolvable fluorescence and negligible quenching. Similarly, some embodiments of the invention may involve ternary or higher orders of combination of colors. With these encoding options, fabrication of >10⁶ uniquely distinguishable barcodes may be achievable by using RE-doped glass fibers.

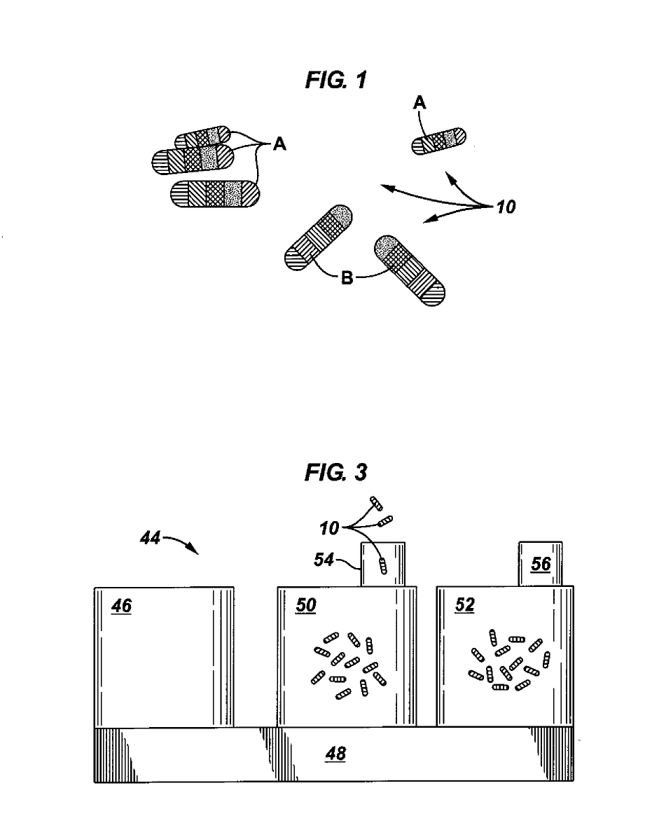

FIG. 1 shows an illustration of particles having microbarcodes in accordance with one embodiment of the invention. As shown, the barcodes are formed in micrometer sized particles 10. In this example, two types of particles A and B are shown, each with a different pattern of the microbarcodes. As noted above, the particles 10 may be illuminated with a suitable UV light source and may be viewed through a suitable filter (e.g., a 420-nm long-pass filter). The large number of combinations that can be encoded onto the particles, their compatibility with solvents, their miniature size, and their ruggedness makes the RE-doped particles 10 highly suitable for various subsurface applications.

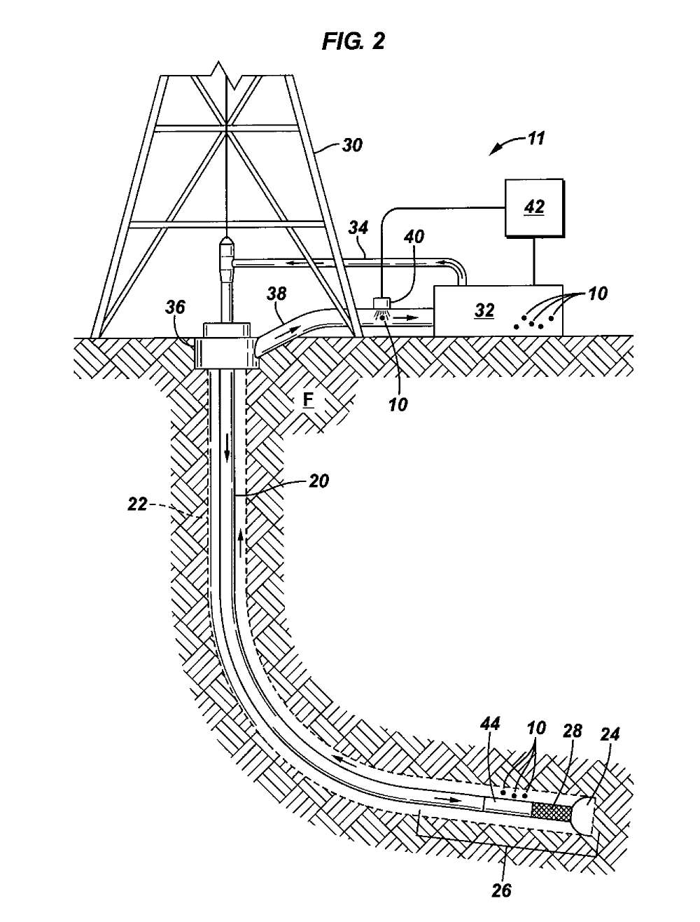

Some embodiments of the invention relate to the use of the microbarcode particles to trace fluids and solids in a subsurface environment and to provide means of communication and monitoring. FIG. 2 shows a schematic illustrating a use of the coded particles in a downhole environment in accordance with one embodiment of the invention. As shown, a system 11 includes a drill string 20, shown disposed within a borehole 22 traversing a subsurface formation F as the hole is cut by the action of the drill bit 24 mounted at the far end of a bottom hole assembly (BHA) 26. The BHA 26 is attached to and forms the lower portion of the drill string 20. BHA 26 may contain a number of devices including various subassemblies 28, which may include those used for measurement-while-drilling (MWD) and/or logging-while-drilling (LWD). Information from the subassemblies 28 is communicated to a telemetry assembly (not shown) in the drill string 20 which conveys the information to the surface as is known in the art (e.g., via pressure pulses through the drilling mud).

At the surface, the system 11 may include a derrick 30 and hoisting system, a rotating system, and a mud circulation system. Although this aspect of the invention is shown in FIG. 2 as being on land, those skilled in the art would recognize that the present invention is equally applicable to marine environments. A mud circulation system pumps drilling fluid down through the central opening in the drill string 20. The mud is stored in mud pit which is part of a mud separation and storing system 32. The mud is drawn in to mud pumps (not shown) which pump the mud through stand pipe 34 and into the Kelly and through the swivel.

The mud passes through drill string 20 and through drill bit 24. As the drill bit grinds the formation into cuttings, the mud is ejected out of openings or nozzles in the bit with great speed and pressure. These jets of mud lift the cuttings off the bottom of the hole and away from the bit, and up towards the surface in the annular space between drill string 20 and the wall of the borehole 22, as represented by arrows in FIG. 2. At the surface the mud and cuttings leave the well through a side outlet in a blowout preventer 36 and through a mud return line 38. The mud return line 38 feeds the mud into the separation and storing system 32, which separates the mud from the cuttings. From the separator, the mud is returned to a mud pit (not shown) for storage and re-use.

According to one embodiment of the invention, coded particles 10 may be disposed in the mud separation and storing system 32, such that they are set for selective release to a subsurface location via the mud flow. A particle detection unit 40 may be coupled into the mud return line 38 and linked to surface equipment 42 comprising a computer, display, recording, and user interface means as known in the art. The detection unit 40 may include a light source (e.g., UV light source), one or more camera devices, and optics to provide appropriate wavelength illumination to the passing particles 10 in order to provide an optical emission such that the individual particle codes may be distinguished.

In accordance with some embodiments of the invention, the detection unit 40 may include a filtering or separating device, such as a centrifuge, to collect the particles 10 for analysis. If the particles 10 comprise a ferromagnetic material, the detection unit 40 may be implemented with means to generate a magnetic field (e.g., permanent magnet or electromagnet) to collect the particles 10 for analysis.

In accordance with some embodiments of the invention, upon resolution of the particle coding, the codes may be matched against a reference database or “code chart.” The detection and identification of the particles 10 may be assisted by the use of a camera that may be used to record images or display images on a screen. In accordance with some embodiments of the invention, the detection unit 40 may comprise a conventional camera configured to record and display images on a screen.

The surface equipment 42 may be configured with a program to process the resolved code data, establish the code matching, track particle travel times, automatically trigger selected particle release, and respectively transmit/receive data/commands to/from remote locations. In accordance with some embodiments of the invention, the surface equipment 42 may be equipped with programs to perform image analysis for particle identification or to calculate the density of particular particles 10.

In accordance with some embodiments of the invention, a simple system can be implemented wherein the particles 10 are initially disposed in the mud manually and captured in the return line 38 (e.g., using a screening filter, magnet means, centrifuge or separator) for processing by rig personnel. The miniature size and structure of the particles 10 may allow them to survive destruction due to the drilling process.

In accordance with other embodiments of the invention, a system may be implemented wherein the particles 10 are set in a release mechanism disposed on the BHA 26, or anywhere along the drill string 20, such that they are selectively or automatically released downhole at a desired depth or when a predetermined event occurs. As illustrated in FIG. 2, the BHA 26 may be implemented with a tool comprising a particle release unit 44.

Referring to FIG. 3, an example of a particle release unit 44 is shown. As shown, the particle release unit 44 may comprise a sensor 46 adapted to sense a subsurface characteristic or condition (e.g., pressure, temperature, fluid composition, flow rates, etc.). Sensors of these types are well known technology, as are the means to power the sensors. Sensor 46 is in communication with a processor 48 which may comprise a number of microprocessors. One or more chambers 50, 52 contain particles 10. Associated with the chambers 50, 52 are release mechanisms 54, 56. The release mechanisms 54, 56 can be activated to selectively release the respective particle(s) 10 under the control of processor 48. The release mechanisms 54, 56 may be configured to release the particle(s) 10 via a forced or pressurized ejection, via direct exposure of the particles to the mud flow, or some combination of these methods or others as known in the art. The release mechanisms 54, 56 may be instructed to release the particles 10 by a program in the processor 48. In this manner, the release mechanisms 54, 56 may be instructed to selectively release their particles 10 when different predetermined thresholds or conditions are determined by the sensor 46, or based on input from other sensors in the system.

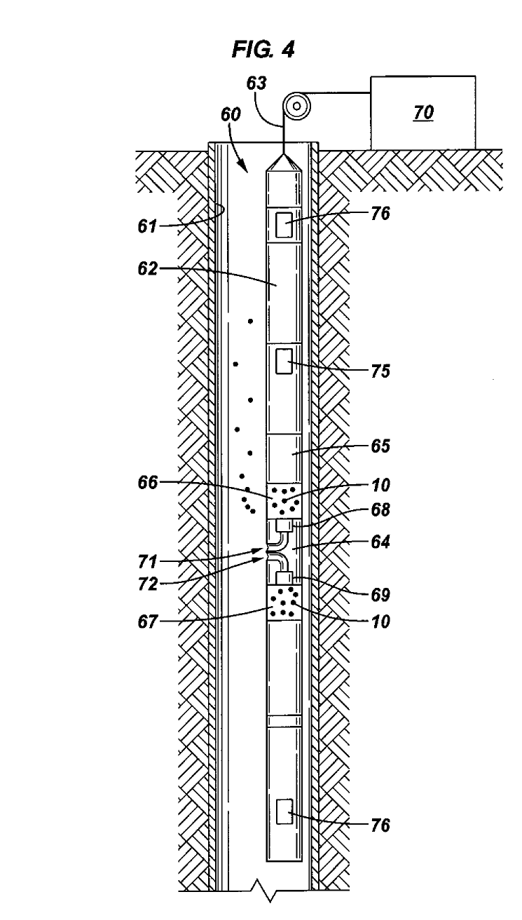

FIG. 4 illustrates another aspect of an embodiment of the invention. As shown in FIG. 4, a system 60 in accordance with one embodiment of the invention may be used in a cased production well 61. A downhole tool 62, having an elongated body, may be suspended from a logging cable or wireline 63. The logging cable or wireline 63 may include one or more conductors that are cooperatively coupled to surface instrumentation 70 for power/signal communication and recordation as a function of time/depth. The tool 62 includes a particle release unit 64 selectively controllable by way of the surface instrumentation 70 or via signals from a processor 65 provided in the tool. The particle release unit 64 may include upper and lower enclosed chambers 66, 67 spatially disposed within the tool 62 body and respectively containing the particles 10 under pressure. The chambers 66, 67 may be configured for selective and repetitive discharge of particles 10 into the well bore.

To control the release of the particles 10 from their respective chambers 66, 67, the release unit 64 may include valves 68, 69 that are coupled to each of the chambers and respectively arranged, upon being opened, to selectively communicate the chambers with discharge ports or laterally-directed orifices 71, 72. The particles 10 may be maintained at elevated pressures which exceed the well bore pressure at the release depth location of the tool 62. As depicted in FIG. 4, the tool 62 may also include one or more sources/sensors 75 comprising conventional measurement means known in the art. It would be appreciated by those skilled in the art that other particle release units may be devised with various types of mechanisms and in different configurations in order to implement aspects of the invention disclosed herein. For example, U.S. Pat. No. 6,125,934 and U.S. Patent Publication No. 20070144737 (both assigned to the present assignee and entirely incorporated herein by reference) describe downhole tools equipped for subsurface tracer release. These tools can be readily implemented for releasing particles 10 of the invention as disclosed herein.

Some embodiments of the invention may also be configured to detect the subsurface fluorescence emissions of the particles 10. Instruments configured to detect fluorescence downhole may be known in the art. For example, U.S. Pat. No. 6,704,109 (assigned to the present assignee and entirely incorporated herein by reference) describes a tool equipped with a probe system configured to illuminate crude oil in the well and detect the emitted fluorescence. Embodiments of the invention can be implemented with similar optical systems such that the particles 10 may be released, irradiated, and observed downhole. The optics and light sources in these conventional systems are already configured to provide illumination of appropriate wavelength, or they can be readily adjusted to output the desired radiation. In one aspect, the tool 62 of FIG. 4 may be implemented with downhole fluorescence detector units 76 mounted at longitudinally-spaced intervals above or below the particle release unit 64. Such embodiments may be used to detect the particles 10 downhole and provide an indication, such as data for example, to the surface instrumentation 70 whenever there is particle movement past a detector 76. Alternatively, a tool (e.g., tool 62) equipped with one or more downhole fluorescence detector units 76 may be used to illuminate and detect particles 10 previously released or affixed to the borehole/casing wall, such as particles 10 disposed in proppant/fracturing compounds and retained in fissures or mudcake. Another aspect of the tool 62 may include an extendable arm (not shown) configured to press or otherwise locate the tool, and the detector units 76, against the borehole or casing wall, as known in the art. Yet another aspect of the tool 62 may be configured with the detector units 76 comprising camera means to configured to capture images of the illuminated particles 10.

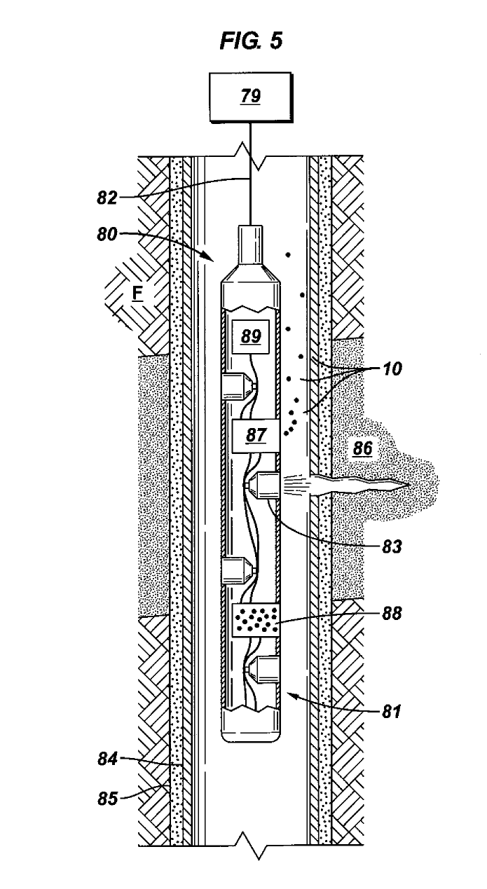

FIG. 5 illustrates another aspect of an embodiment of the invention. As shown in FIG. 5, a system 80 may include a perforation tool incorporating releasable particles 10. A perforation gun 81 is suspended from a wireline 82 linked to surface equipment 79 via conventional deployment hardware. The perforation gun 81 essentially comprises a plurality of shaped charges mounted on the gun frame. One of the charges 83 shown in FIG. 5 is illustrated as having been fired. The firing charge may produce a perforation through the casing 84 and cement 85 into the reservoir region 86 in the subsurface formation F. One or more particle release units 87, 88 may be provided to detect the firing of each shaped charge and release the particles 10. In FIG. 5, particle release unit 87 is shown releasing particles 10. Another aspect of an embodiment of the invention may be implemented with the particles 10 incorporated into the charges themselves such that they are automatically released when the charge is fired (not shown). As with the other systems of the invention, these aspects may be configured for selective release of the particles 10 from the surface and/or via processor means 89 disposed in the gun 81. One use of this system 80 may be to provide positive communication to the surface that a charge was properly fired.

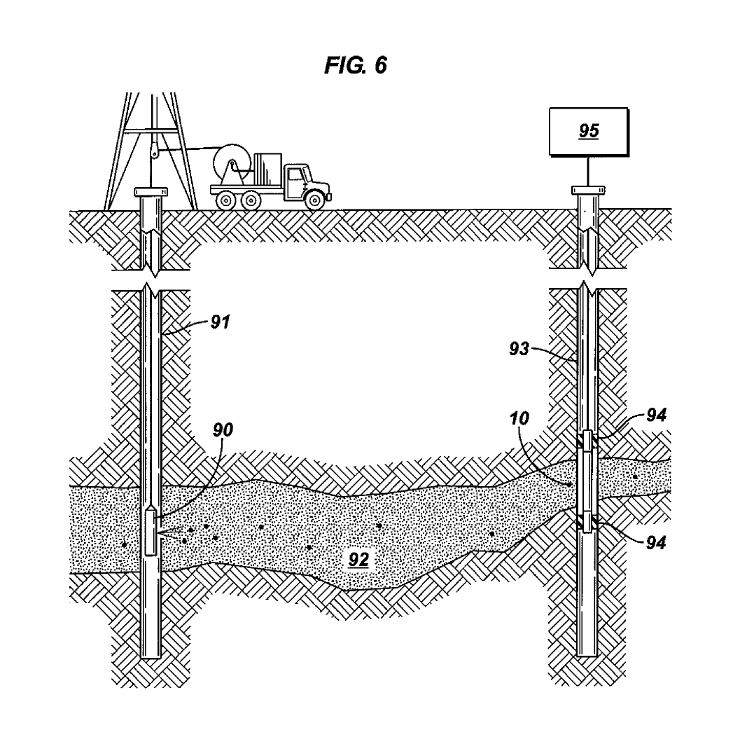

FIG. 6 illustrates another embodiment of an aspect of the invention. In these embodiments, the coded particles 10 may be used for cross-well applications. As shown in FIG. 6, a tool 90 containing the particles 10 is disposed in a first well 91 and activated to release the particles at a desired time and depth. The first well 91 traverses an oil (or water) zone 92 that extends across a field and is traversed by a second well 93. The second well 93 is shown comprising a pair of conventional packers 94 set in place within the well in order to restrict inflow to the well to within a specific depth range including the zone 92. Surface equipment 95 at the second well 93 is used to monitor and record any particles 10 detected at the second well. These data may be correlated to the depths and times of particle 10 release at the first well 91, or in combination with particle release from multiple other wells in the field (not shown). The particle-equipped tool 90 may be any downhole instrument implemented with a particle release mechanism such as those disclosed herein. This aspect of an embodiment of the invention allows one to perform various operations, including but not limited to, tracking and monitoring specific well production, cross-flow monitoring, completion status/performance checks, and reservoir management.

The above-described examples offer a variety of applications for the coded particles 10. In addition to, and further elaborating on, the previously disclosed applications, uses of the coded particles 10 for subsurface applications may include, but are not limited to:

Mud logging—The use of differently coded particles added to the drilling mud at different times provides different types of information:

1. Circulation time at specific time slots. The travel time of different particles may be logged. The time between the release and the detection of the particles may be measured, as well as the travel time between two or more established locations.

2. Mud loss detection. A dip in the concentration of a given tagged particle in the mud may indicate greater loss of drilling fluid at a particular depth.

3. Kick location. A surge in the concentration of given tagged particles in the mud may indicate that that zone is starting to produce.

4. Mud cake formation estimation.

Mud cake tagging—The use of differently coded particles added to the drilling mud at different times may tag the mud cake as a function of depth that may be correlated with the drilling depth. This provides for:

1. Correlating drilling depth and wireline depth. This may be done by sampling the mud cake at certain depths.

2. Cement placement identification by analyzing the displaced mud.

3. Acidizing job/Acid injection monitoring. By analyzing the particles returning from the mud cake one may locate where the treatment is effective.

4. Perforating monitoring. Produced particles may be analyzed to correlate the position of perforations.

5. Clean up treatment monitoring. The amount and type of debris may be estimated using tagging with the particles.

Drill bit communication—In cases where mud pulse telemetry cannot be used, a sub near the drill bit (e.g., unit 44 in FIG. 2) may selectively release a combination of coded particles into the mud to convey information from the drill bit to the surface.

Proppant placement monitoring—Different types of coded particles may be added to the proppant in the fracturing fluid at different times. The concentration of the returned or produced particles of each type may give the efficiency of the fracturing operation.

Gravel pack monitoring—Different types of tagged particles may be added to the gravel at different times during the gravel packing operation. The effectiveness of the placement at different stages of the operation may be monitored by analyzing the concentration of the different particle types returned to the surface. The operation may also be monitored during production and for any sand production. The monitoring may be used to identify which region of a gravel pack has failed, for example.

Completion operation monitoring—A sub near a given element of the completion (packer, flow control valve, latching mechanism, etc.) could selectively release a combination of tagged particles into the produced fluid to convey information to the surface. Monitoring of the fluid could reveal information about the status of the particular device.

Well treatment monitoring—Particles may be mixed with solid acids or other compounds in order to track/monitor completion operations.

Flow measurement (Production Logging Techniques, slick line, permanent)—The release of tagged particles into the flow may be used to obtain flow velocity. In such aspects, the particles' surface may be treated as known in the art to increase their affinity to a given fluid when multi-phase flows are measured.

Field-scale monitoring—Particle release may be used for injection identification/monitoring, acid injection monitoring, water front/back allocation, diversion detection, multi-zone stimulation.

Gas market measurements—Particles may be used to track fracturing fluids in tight gas shale ore.

Geothermal services—Particles may be used to determine individual reservoir temperatures and how the heat is being lost upon subsequent transportation to the surface.

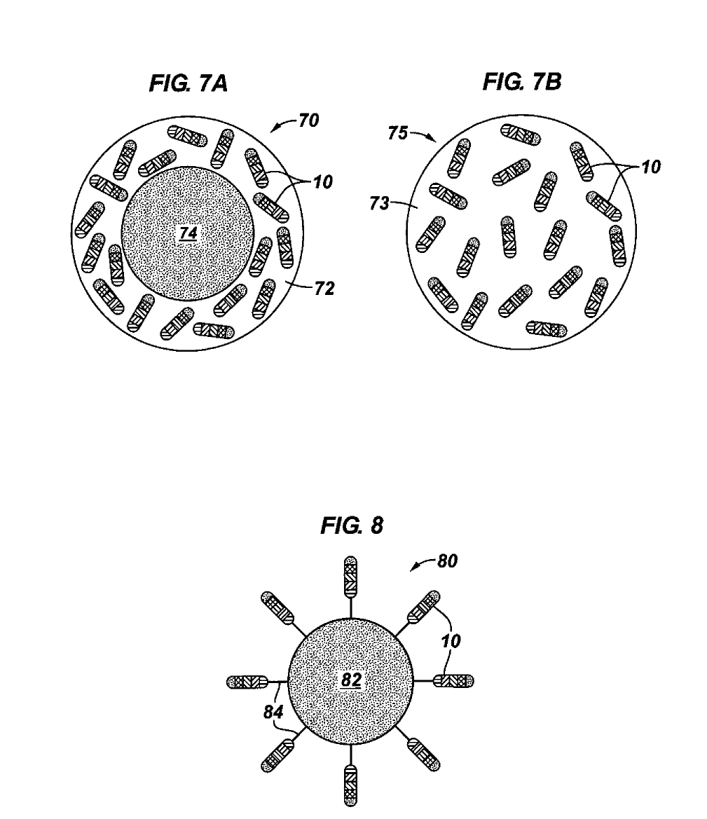

As shown above, the coded particles 10 may be made of materials such as silicon oxide, germanium oxide, aluminum oxide, boron oxide, glass, crystal, ceramic, polymer, Zirconium, or a ferromagnetic material. In some embodiments, the coded particles may be used on their own or mixed with other materials (e.g., fluids). In addition, the coded particles may be physically tethered or bound to other objects (such as proppants). For example, FIG. 7A shows a modified proppant 70, in which the main body (proppant) 74 may be coated by a layer of polymer or composite materials in order to form an envelope or capsule 72 over the proppant body 74. The coating that forms the envelope or capsule 72 may comprise a plurality of coded particles 10. Furthermore, the envelope or capsule 72 may be solvent soluble or solvent insoluble, depending upon the specific application of use.

Alternatively, the coded particles 10 may be mixed with the materials that form the objects (e.g., proppants). For example, FIG. 7B shows a modified proppant 75, which may be made of a material 73 (e.g., ceramic, composite or polymer materials). A plurality of the coded particles 10 are mixed into the material 73 before they are made into proppants 75. The resulting combination may be referred to as coded proppants.

FIG. 8 shows another example of how to attach the coded particles 10 to an object 82 (e.g., such as a proppant). As shown in FIG. 8, a coded object 80 may be prepared by linking one or more coded particles 10 to the object 82. The attachment may be via a linker 84, which may be made of any suitable material. One example of such a linker 84 is a polymer (e.g., thermoplastic resin or thermoset resin) that encapsulates the object 82.

Such modified objects (e.g., proppants) may be used in various downhole applications. For example, the particle-imbedded proppant 70 may be mixed with fracturing fluids to fracture the subsurface formations. These coded proppants 70 may be lodged in the fractures. If the coating 72 is made of an oil-soluble material, when a zone starts to produce hydrocarbons, the coded particles 10 may be released from the coded proppants 70 and subsequently detected in the well fluid. Alternatively, if the coating 72 is made of a water-soluble material, when a zone starts to produce water, the coded particles 10 may be released from the coded proppants 70 and subsequently detected in the well fluid. The detection and identification of particles 10 may therefore help in determining the locations of downhole hydrocarbon or water production zones.

For different location, zone, or fluid identifications, different coded particles 10 may be used. For example, differently coded particles may be placed in one or more oilfield objects or regions. The particles 10 may be selectively released after one or more downhole events trigger the release. Subsequent detection and identification of the different coded particles 10 may allow more detailed analysis of these downhole events based upon the different patterns of fluorescence emission. For example, the differently coded particles 10 may be placed in water-soluble containers (or capsules) at various junctions of multilaterals wells. The detection of the particles 10 may help in identifying the water producing junctions in the multilaterals. The analysis may further allow for the water producing zone and/or junctions in the multilaterals to be shut in/squeezed off.

General testing—Particles 10 may be sent from the surface or selectively released downhole to test the operation of downhole instruments and/or to determine/monitor downhole conditions. Particles 10 may be added to the mud, cement, acid, injection fluid, produced fluid, fracturing fluid, proppant, treatment fluid, gravel, etc. The location of an event may be determined by the type and concentration of particles 10 detected. Different particle sizes may be used in combination to perform any of the operations disclosed herein.

For example, the use of different sized particles 10 may allow for the determination of the size of a fracture, fault, porous medium, etc., that serves as a conduit for the fluids and the associated particles 10.

The multiple coded particles 10 may also be used for multilayer wells. For example, specific coded particles 10 may be added to the fracturing fluids that are used to fracture each individual layer. If the size of particles 10 are small enough to flow through the proppants, detection of the different coded particles 10 may help the user determine the conditions of zones that are contributing to production.

In another embodiment, different coded particles 10 may be placed in the cement surrounding the casing at different depths. The particles 10 could be released after perforations. Subsequent detection of specific coded particles 10 may help the user determine the location of the perforations.

In another embodiment, specifically coded particles 10 may be added to the substances inside one or more injectors. For example, these particles 10 may be used with a steam injector to enhance oil recovery. The different coded particles 10 may be added to different steam injectors. The particles 10 may be released after injection. The later detection and identification of specific coded particles 10 may help the user monitor the individual steam injections.

In another embodiment, specifically coded particles 10 may be added to acid in acid injectors to fracture the formations (e.g., limestone reservoir rock formations). The detection and identification of specific coded particles 10 may help the user monitor the progress and effectiveness of acid injections.

In another embodiment, specifically coded particles 10 may be added to different injection fluids. The detection of particles 10 may help the user determine fluid movement, fluid allocation, and fluid assurance in the reservoir.

In another embodiment, specifically coded particles 10 may be prepared to have different densities such that the different density particles 10 with would float in corresponding different types of fluids, e.g., oil or water. The detection of the different density particles 10 would allow the user to monitor the corresponding type of fluid movement in the reservoirs.

In another embodiment, specifically coded particles 10 may be placed in the triggers at various junctions of multilaterals. Particles 10 may be released when the triggers make contact with a downhole tool. Detection of the particles 10 may identify the junctions that have been entered by the tool.

In another embodiment, different coded particles 10 may be added to different wells or reservoirs that may become comingled during production. Different coded particles could flow with the fluids and may get comingled. Detection and identification of different coded particles 10 may help the user allocate the contributions of different sources to the comingled fluid production. Similarly, different coded particles 10 may be used to trace the contributions of various fluids from different pipe lines that comingle.

In another embodiment, specifically coded particles 10 may be added to drilling fluids (or drilling mud) at different depths. Thus, the particles 10 may become imbedded in filter cakes. Mud cake loss in a borehole may indicate deteriorating integrity of a well that may eventually lead to well collapse. Therefore, the detection of mud cake loss may allow an operator to take corrective action in order to fix the problem. If removal of mud cakes is desired, the embedded particles 10 may also help the user ascertain that the mud cakes are removed or cleaned up.

Loss of proppant (or proppant flowback) from a fracture may cause production decline and damage to production equipment. Therefore, early detection of proppant flowback is important in the maintenance of high conductivity of a fracture and in order to improve long-term production. Embodiments of the tagged proppants 70, 75, and 80, shown in FIGS. 7A, 7B and 8, for example, can be used for this purpose. The tagged proppants 70, 75, and 80 may be added to a fracturing fluid. Detection of the particles 10 may help in monitoring for events of proppant flowback.

In another embodiment, different coded particles 10 may be encapsulated in solvent soluble enclosures (envelopes) and placed at multilateral junction. Particles 10 may then be released when the solvent soluble materials make contact with the solvents, which may be pumped through when a multilateral junction is re-entered. Identification of the particles 10 may help the user identify which multilateral junction has been entered.

In another embodiment, specifically coded particles 10 may be added to inhibitor fluids in a flow assurance treatment. The particles 10 may flow back to the releasing unit. Detection of particles 10 may help determine the rate of inhibitor return.

In another embodiment, specifically coded particles 10 may be placed at various depths in an extended reach well. The particles 10 would be released when production starts. Detection of the particles 10 may help to indicate whether production is from the toe, heal or intermediate portion of the wellbore.

In another embodiment, specifically coded particles 10 may be added to drilling fluids. Thus, the particles 10 may be imbedded into filter cakes when PowerDrive® pads are engaged against the wellbore walls at various intervals, e.g., every 5 to 10 meters. Detection of the particles 10 may help in identifying the different locations inside a downhole.

In another embodiment, specifically coded particles 10 may be immobilized inside the solid acid materials, e.g., polymers, in AcidMAX®. The particles 10 may be released when the solid acid materials are converted to acid form. Detection of the returned particles 10 may indicate that a conversion of the solid polymers to an acid form has taken place.

In another embodiment, specifically coded particles 10 may be added to fluids flowing through control valves. The time between the release of the particles 10 from each valve and the detection of the return of the particles 10 may be used in determining the flow rate from each valve. Accordingly, the flow control valves may be more accurately controlled and optimized.

In another embodiment, specifically coded particles 10 may be immobilized in pillars (fiber slug). The particles 10 may be released if the integrity of the pillar formation has been compromised. Detection of the particles 10 in well fluid may therefore indicate the location and identity of a compromised pillar formation.

In another embodiment, specifically coded particles 10 may be immobilized with respect to various outside sections or portions of a casing. The particles 10 may be released as a result of leaks developing in the casing. Detection of the particles 10 may help in identifying the location of a casing leak.

Modification of formation wettability may cause damage to the well by changing the well's relative permeability to water, oil, or gas, thereby affecting well productivity. Accordingly, early detection of formation wettability may help in the prevention of such damage. In one embodiment, specifically coded particles 10 may be immobilized in various production zones. The particles 10 may either adhere or be released when there is a change in the formation wettability. For example, the particles 10 may be designed to adhere to an oil-wet formation. A decreased detection of particles 10 in such a case may indicate an oil-wet formation. When the formation changes from oil-wet to water-wet, however, the particles 10 may be released from the formation. As a result, an increased detection of the particles 10 may indicate a change in the formation wettability from oil-wet to water-wet. Conversely, the particles 10 may be designed to stick to a water-wet formation. A decreased detection of the particles 10 in this case may indicate a water-wet formation. However, when the formation wettability changes from water-wet to oil-wet, the particles 10 may be released from the formation. As described, detection of the particles 10 may help determine wettability changes in a formation.

In another embodiment, specifically coded particles 10 may be added to water (or CO₂) flooding in an enhanced oil recovery operation. Detection of the particles 10 may help to indicate the water (or CO₂) flooding patterns. The datasets resulting from the detections may then be combined with 4D seismic technology. Changes occurring in the reservoir as a result of hydrocarbon production or the injection of water (or CO₂) into the reservoir may be determined by comparing repeated datasets.

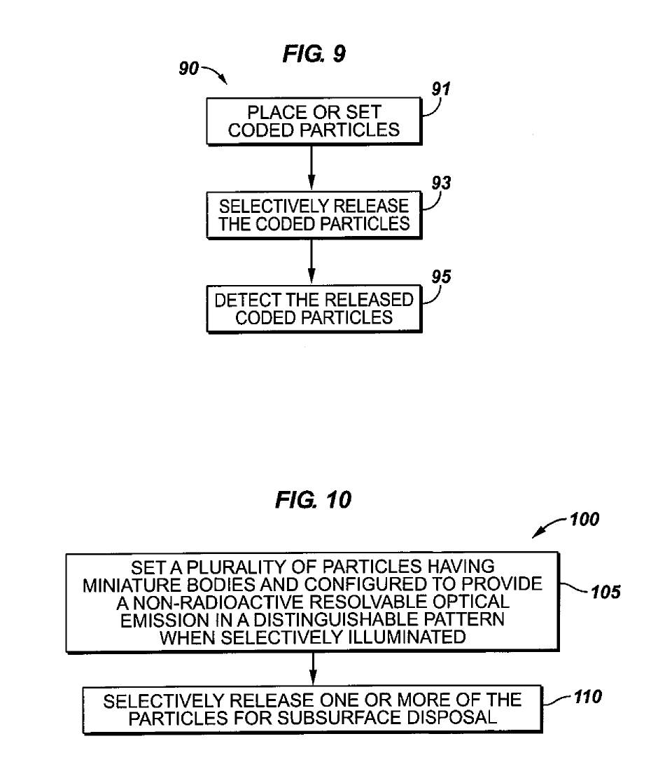

FIG. 9 shows a flow chart illustrating an embodiment of a method of the invention. As shown, an illustrative method of using the coded (tagged) particles 90 may include initially setting or placing the coded particles (step 91). For example, the coded particles may be placed into a well, a subsurface formation or a subsea formation.

The coded particles may be selectively released. For example, the coded particles may be released by a trigger mechanism (step 93). The triggering event or mechanism may include any described above, such as production of oil, water, etc.

Further, the released coded particles may be identified (step 95) and in some cases quantified. The identification of the coded particles may provide information regarding the occurrence of the individual trigger events. In some cases, the detection of the coded particles may provide information regarding the associated well flow composition, formation characteristics, etc.

While various illustrative embodiments of the invention have been described with respect to a limited number of exemplary embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A tagged object, comprising:
a main body; and
a plurality of coded particles,
wherein each coded particle having a miniature body and configured to provide a resolvable optical emission pattern when illuminated, and
wherein the plurality of coded particles are immobilized to the main body.

2. The tagged object of claim 1, wherein the plurality of coded particles are immobilized to the main body by being embedded in the main body.

3. The tagged object of claim 1, wherein the plurality of coded particles are immobilized to the main body by a coating covering the main body.

4. The tagged object of claim 3, wherein the coating is insoluble in a selected solvent.

5. The tagged object of claim 1, wherein the plurality of coded particles are immobilized to the main body by a tether to the main body.

6. The tagged object of claim 5, wherein the tether is soluble in a selected solvent.

7. The tagged object of claim 1, wherein the tagged object is a proppant.

8. The tagged object of claim 1, wherein the miniature body of each of the plurality of coded particles is made of a material selected from the group consisting of silicon oxide, germanium oxide, aluminum oxide, boron oxide, glass, crystal, ceramic, polymer, Zirconium, a ferromagnetic material, and a mixture thereof.

9. A system for tagging, comprising a plurality of tagged objects, wherein each of the plurality of tagged objects comprises:
a main body; and
a plurality of coded particles,
wherein each coded particle having a miniature body and configured to provide a resolvable optical emission pattern when illuminated,
wherein the plurality of coded particles are immobilized to the main body and wherein the plurality of tagged objects each have a different optical emission pattern.

10. The system of claim 9, wherein the plurality of coded particles are immobilized to the main body by being embedded in the main body.

11. The system of claim 9, wherein the plurality of coded particles are immobilized to the main body by a coating covering the main body.

12. The system of claim 11, wherein the coating is insoluble in a selected solvent.

13. The system of claim 9, wherein the plurality of coded particles are immobilized to the main body by a tether to the main body.

14. The system of claim 13, wherein the tether is soluble in a selected solvent.

15. The system of claim 9, wherein the plurality of tagged objects are proppants.

16. The system of claim 9, wherein the plurality of tagged objects have different densities.

17. A method for performing oilfield monitoring, comprising:
disposing different types of tagged objects at different locations, wherein the different types of tagged objects each comprise a plurality of coded particles, each of which has a miniature body containing rare earth elements to produce a unique optical emission pattern when illuminated;
allowing an event to trigger a release of one of the different types of tagged objects from one of the different locations, and
identifying the released tagged objects by unique optical emission patterns to determining an occurrence location of the event.

18. The method of claim 17, wherein the different locations are different subsurface formation layers.

19. The method of claim 17, wherein the different locations are different pipelines.

20. The method of claim 17, wherein the different locations are different wells penetrating a subsurface formation.

21. The method of claim 17, wherein the disposing is by placing the different types of tagged objects in fracturing fluids and formation fracturing operations, and wherein the tagged objects are tagged proppants.

22. The method of claim 17, wherein the disposing is by placing the different types of tagged objects in drilling fluids to label mud cakes at the different locations.

23. The method of claim 17, wherein the disposing is by placing the different types of tagged objects in cement surrounding a casing.

24. The method of claim 17, wherein the disposing is by placing the different types of tagged objects in one or more steam injectors and performing steam injection operations.

25. The method of claim 17, wherein the disposing is by placing the different types of tagged objects in one or more triggers positioned at the junctions of a multilateral well
